(19) United States
(12) Patent Application Publication
(10) Pub. No.: US 2011/0112623 A1
(43) Pub. Date: May 12, 2011

(54) System and Method for Placing a Coronary Stent at the Ostium of a Blood Vessel

(76) Inventor: Richard A. Schatz, San Diego, CA (US)

(21) Appl. No.: 12/940,877

(22) Filed: Nov. 5, 2010

Related U.S. Application Data
(60) Provisional application No. 61/259,871, filed on Nov. 10, 2009.

(51) Int. Cl. A61F 2/84

(52) U.S. Cl. 623/1.11

(57) ABSTRACT

A system and method are provided that use a balloon/stent catheter for placing a stent at the ostium of a blood vessel. Included is a barrier member that is attached to the catheter at a location proximal the balloon. In use, the barrier member is reconfigured, prior to insertion of the balloon/stent portion of the catheter through the ostium. With this reconfiguration, a barrier (array) is established that limits advancement of the catheter into the blood vessel to ensure proper placement of the stent at the ostium.

This application claims the benefit of U.S. Provisional Patent Application Ser. No. 61/259,871, filed Nov. 10, 2009.

### FIELD OF THE INVENTION

The present invention pertains generally to intravascular catheter systems. More particularly, the present invention pertains to balloon catheters that are used to place stents in the vasculature of a patient. The present invention is particularly, but not exclusively, useful as a stent delivery system for placing a stent at the ostium of a blood vessel, wherein the catheter incorporates a barrier (array) that limits insertion of the balloon/stent through the ostium of the blood vessel, to ensure a proper placement of the stent at the ostium.

### BACKGROUND OF THE INVENTION

After an angioplasty procedure, it is well known that there is always the possibility of a restenosis or, perhaps, a collapse of the weakened blood vessel at the site of the procedure. To help minimize either of these possibilities, and to hopefully avoid them altogether, it has become a standard practice to place stents at the angioplasty site after the procedure. The purpose here is to give added strength to the wall of the blood vessel that may otherwise be lacking as a result of the angioplasty procedure. Typically, the placement of a stent in the vasculature of a patient (i.e. in a blood vessel) is accomplished using a balloon catheter. More specifically, after a stent has been positioned (crimped) onto the balloon of a balloon catheter (i.e. to create a balloon/stent catheter) the stent is advanced into the vasculature and to the site where it is to be placed. The balloon is then inflated to expand the stent, for placement of the stent against the wall of the blood vessel. Once the stent has been placed, the balloon is then deflated and the balloon catheter is withdrawn from the vasculature, leaving the stent in place.

Under conditions where the stent placement site is located along the length of a blood vessel, placement of the stent is relatively straightforward. It is, however, an entirely other matter when the intended stent placement site is at the ostium of a blood vessel. In this latter case, not only does the stent need to be properly inserted into the ostium, its insertion must be limited to ensure a proper placement of the stent. Importantly, the complete stent needs to be placed at the ostium. Stated differently, there should not be a partial insertion that leaves part of the stent hanging outside the blood vessel, in the aorta, nor should the stent be inserted beyond the ostium, deeper into the blood vessel. Heretofore, a proper placement of the stent has relied solely on visual information provided by well-known imaging techniques, such as fluoroscopy.

With the above in mind, it is an object of the present invention to provide a system and method for placing a stent at the ostium of a blood vessel that gives a tactile sensation for the task, in addition to visual information, to ensure the proper placement of a stent at the ostium. Another object of the present invention is to provide a system and method for placing a stent at the ostium of a blood vessel that prevents either too much overhang in the aorta or a deep placement of the stent into the blood vessel that causes the stent to miss the ostium. Yet another object of the present invention is to provide a system and method for placing a stent at the ostium of a blood vessel that is relatively simple to manufacture, is easy to use, and is comparatively cost effective.

### SUMMARY OF THE INVENTION

A delivery system for placing a stent at the ostium of a blood vessel includes a catheter having an inflatable balloon. The balloon is mounted at the distal end of the catheter, and a stent is positioned (crimped) onto the balloon. Thus, a balloon/stent catheter is created. Additionally, a ring is affixed to the catheter shaft at a location proximal to the balloon, and a barrier member is attached to the ring. In overview, the barrier member can be manipulated to ensure a proper placement of the stent at the ostium of a blood vessel in the vasculature of a patient.

Structurally, the barrier member includes a plurality of deflectable extension members that are biased to flare outwardly from the shaft in a radial direction to create a pattern (array). To do this, each of the extension members is moveable in concert with the other extension members between a first configuration, wherein all of the extension members are aligned substantially parallel to the axis of the catheter, and a second configuration wherein all of the extension members are biased to extend substantially perpendicular to the axis. It is in this second configuration, that the plurality of extension members project in a radial direction from respective locations at the proximal end of the stent. Thus, they establish a structural array that can be operationally used as a barrier, to limit advancement of the catheter into the vasculature.

In greater detail, the barrier member includes a base portion having a proximal end that is attached to the ring. The plurality of deflectable extension members that are disclosed above, are integral with the distal end of this base portion. Further, as implied above, when the barrier member is in its second configuration, deflectable extension members are biased into an array. More specifically, this array assumes a predetermined pattern, such as a web-like pattern, a fan-like pattern, or a pattern of elongated extension arms. Also, the base portion of the barrier member may be constructed in a manner that will allow it to be deformable when the balloon is inflated.

The system may also include a guiding catheter that cooperates with the balloon/stent catheter to perform at least two functions. One, is to advance the balloon/stent catheter through the vasculature. The other is to manipulate the barrier member of the balloon/stent catheter. Structurally, the guiding catheter is coaxial with the balloon/stent catheter, and is positioned over the balloon/stent catheter for axial movement relative to the catheter. Within this structure, the guiding catheter can alternately confine the array inside the guiding catheter with the array in its first configuration (i.e. advancement through the vasculature), or release the array into its second configuration for placement of the balloon/stent catheter at an ostium of a blood vessel.

Additional features of the system include an extracorporeal inflation pump that is connected to the proximal end of the catheter, and is in fluid communication with the balloon for selectively inflating the balloon to deploy the stent. Also, a guide wire can be pre-positioned in the vasculature of a patient with the guide wire extending into the ostium of the blood vessel. The guide wire can then be engaged with the balloon/stent catheter for advancing the catheter through the vasculature to the ostium. As envisioned for the present invention, the barrier member is preferably made of cobalt, chromium, platinum, nitinol or stainless steel.

For an operation of the present invention, a guide wire is pre-positioned in the vasculature of a patient, with the guide wire extending into the ostium of the blood vessel. The balloon/stent catheter is then advanced over the guide wire, inside the guiding catheter. This continues until the distal end of the balloon/stent catheter is near the ostium. At that point, either the balloon/stent catheter is advanced through the guiding catheter in a distal direction, or the guiding catheter is withdrawn in a proximal direction to reconfigure the barrier member from its first configuration, and into its second configuration. The balloon/stent catheter is then further advanced, with the array in the second configuration, for use of the barrier to prevent an advancement of the catheter into the ostium, after the stent has been properly positioned in the ostium. The balloon is then inflated to deploy and place the stent in the ostium. Next, the balloon is deflated, and the catheter, less the stent, is withdrawn into the guiding catheter. This withdrawal also returns the barrier member to its first configuration. The system is then withdrawn from the vasculature.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a presentation of the system of the present invention shown in its intended operational environment;

FIG. 2 is a perspective view of the system with portions broken away for clarity;

FIG. 3A is a cross-section view of the distal portion of the balloon/stent catheter of the present invention as seen along the line 3-3 in FIG. 2, with the guiding catheter positioned over the balloon/stent catheter to confine the barrier member of the catheter in its first configuration;

FIG. 3B is a cross-section view of the distal portion of the balloon/stent catheter as in FIG. 3A, with the guiding catheter withdrawn to bias the barrier member into its second configuration;

FIG. 3C is a cross-section view of the distal portion of the balloon/stent catheter as in FIG. 3B, with the stent inserted into a blood vessel and the balloon inflated to place the stent in the ostium of the blood vessel;

FIG. 4A is a side view of the barrier member of the present invention with its base portion configured for deformation;

FIG. 4B is a side view of the barrier member of the present invention with its base portion alternately configured for deformation;

FIG. 4C is a side view of the barrier member of the present invention with its base portion further alternately configured for deformation;

FIG. 5A is an alternate embodiment of the array of the barrier member as seen from along the axis of the catheter; and

FIG. 5B is another alternate embodiment of the array of the barrier member as seen from along the axis of the catheter.

### DESCRIPTION OF THE PREFERRED EMBODIMENTS

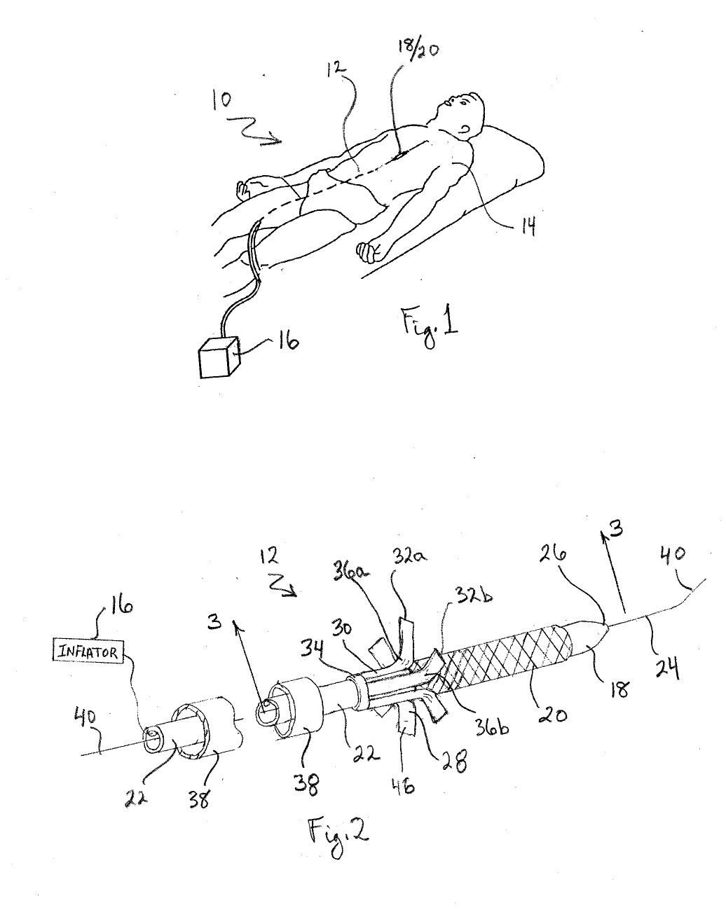

Referring initially to FIG. 1 a system for delivering a stent to the ostium of a blood vessel in accordance with the present invention is shown and is generally designated 10. As shown, the system 10 includes a balloon/stent catheter 12 that is being advanced into the vasculature of a patient 14. Once the catheter 12 has been properly placed in the vasculature, an inflator 16 is activated to inflate a balloon 18. With this inflation of the balloon 18, a stent 20, that is positioned (crimped) onto the balloon 18, will expand. The stent 20 will then be left at the ostium of a blood vessel after the catheter 12 is withdrawn from the vasculature. Structural details of the balloon/stent catheter 12 will be best appreciated with reference to FIG. 2.

In FIG. 2 it will be seen that the balloon/stent catheter 12 includes a shaft 22 defining a longitudinal axis 24. For the present invention, the balloon 18 is affixed to the shaft 22 at a location immediately proximal the distal end 26 of the shaft 22. Further, FIG. 2 shows that the catheter 12 includes a barrier member 28 which is affixed to the shaft 22 at a location immediately proximal the balloon 18. As shown, the barrier member 28 includes a base portion 30 with a plurality of deflectable extension members 32 projecting therefrom. The extension members 32a and 32b shown in FIG. 2 are only exemplary.

Structurally, the base portion 30 of the barrier member 28 is mounted on a ring 34 that is affixed to the shaft 22 of the balloon/stent catheter 12. Within this arrangement, it is an important aspect of the barrier member 28 that each extension member 32 is biased at a bending region 36 which is intermediate the base portion 30 and the extension members 32. More specifically, in their relaxed state, the extension members 32 are biased to assume the configuration shown in FIG. 2. In this configuration, the extension members 32 extend outwardly, in a radial direction from the axis 24. The bending regions 36a and 36b shown for respective deflection extension members 32a and 32b are exemplary. Preferably, the barrier member 28 is made of cobalt, chromium, platinum, nitinol or stainless steel.

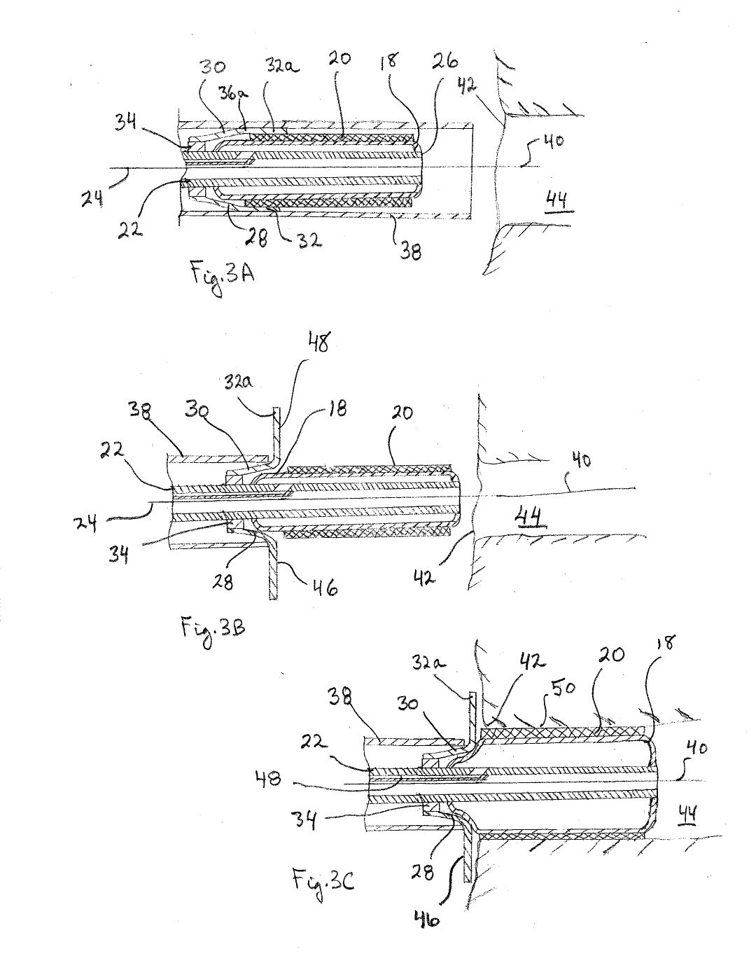

Still referring to FIG. 2, it will be seen that system 10 may also include a guiding catheter 38 and a guide wire 40. For the system 10, the purpose of this guiding catheter 38 is actually two-fold. For one, the guiding catheter 38 can be used to advance the balloon/stent catheter 12 through the vasculature of patient 14 to the ostium 42 of a blood vessel 44. For this purpose, a configuration of the balloon/stent catheter 12 and its barrier member 28, in combination with the guiding catheter 38, is shown in FIG. 3A. More specifically, in FIG. 3A it will be appreciated that all of the extension arms 32 are forced by the guiding catheter 38 to align substantially parallel to the axis 24. As shown in FIG. 3B, however, when either the stent/balloon 20/18 is advanced in a distal direction out of the guiding catheter 38, or when the guiding catheter 38 is withdrawn in a proximal direction, the extension arms 32 of barrier member 28 are biased into a flared configuration. In this flared configuration, they extend outwardly in a radial direction substantially perpendicular to the axis 24 to establish an array 46.

FIG. 3C indicates that once the guiding catheter 38 has been withdrawn from over the stent/balloon 20/18, and the barrier member 28 consequently assumes its flared configuration to present the array 46, the stent/balloon 20/18 can then be inserted into the ostium 42 of blood vessel 44. Importantly, during this insertion, the array 46 that is established by the now-deflected extension members 32 of the barrier member 28 will act as a barrier to limit insertion of the catheter 12 into the blood vessel 44. Moreover, because it is radiopaque, in addition to visual clues (i.e. fluoroscopy), the array 46 will give the user of the catheter 12 a tactile sensation of contact with the ostium 42.

FIG. 3C also indicates that after the stent/balloon 20/18 has been inserted into the blood vessel 44, the inflator 16 can be activated to inflate the balloon 18, via an inflation tube 48 that connects the balloon 18 in fluid communication with the inflator 16. When the balloon 18 has been inflated, the stent 20 is expanded and pressed against the wall 50 of the blood vessel 44. Importantly, due to the constraint on advancement of the catheter 12, as imposed by the array 46 of barrier member 28, the stent 20 is properly positioned at the ostium 42 of the blood vessel 44. The balloon 18 can then be deflated and retracted into the guiding catheter 38 (see FIG. 3A). The catheter 12, less stent 20, and the guiding catheter 38 can then be withdrawn from the vasculature.

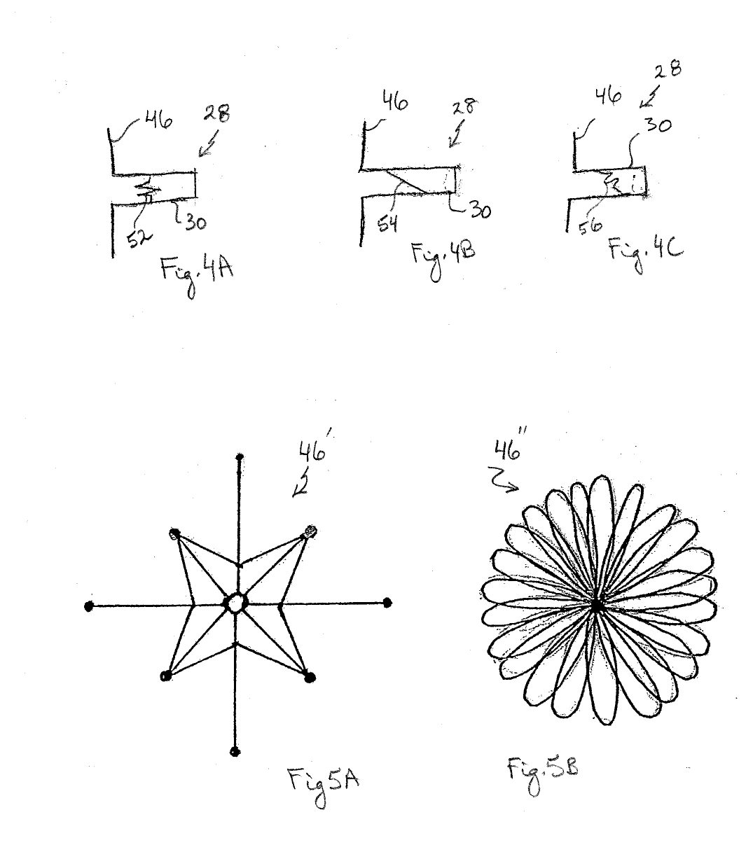

Referring now to FIGS. 4A, 4B and 4C, several structural variations for the base portion 30 of barrier member 28 are shown. Specifically, these different structures are intended to allow the base portion 30 to be at least somewhat deformed during an inflation of the balloon 18. To do this, FIG. 4A shows the base portion 30 with a “Z” shaped slit 52; FIG. 4B shows it with an “angled” slit 54; and FIG. 4C shows an “S” shaped slit 56. Further, in addition to the preferred configuration for array 46 shown in FIG. 2, wherein the extension members 32 flare radially outward from the axis 24, FIG. 5A shows a variation wherein the array 46′ is shown as a web-shaped pattern. As another alternative embodiment, FIG. 5B shows an array 46″ having a fan-shaped pattern.

While the particular System and Method for Placing a Coronary Stent at the Ostium of a Blood Vessel as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A delivery system for placing a stent at the ostium of a blood vessel, the system comprising:
a catheter having a shaft with a proximal end and a distal end, and defining a longitudinal axis;
an inflatable balloon mounted at the distal end of the catheter;
a stent positioned on the balloon, with the stent having a proximal end and a distal end;
a ring affixed to the catheter shaft at a location proximal to the balloon;
a barrier member attached to the ring, wherein the barrier member includes a plurality of deflectable extension members, with the plurality being collectively moveable between a first configuration wherein all of the extension members are aligned substantially parallel to the axis of the catheter, and a second configuration wherein all of the extension members are biased to extend substantially perpendicular to the axis from respective locations adjacent the proximal end of the stent to establish an array for use as a barrier; and
a means for advancing the catheter with the array in the second configuration for use of the barrier to prevent any further distal advancement of the catheter after the stent has been positioned in the ostium for placement of the stent in response to an inflation of the balloon.

2. A system as recited in claim 1 further comprising an extracorporeal inflation means connected to the proximal end of the catheter in fluid communication with the balloon for selectively inflating the balloon to deploy the stent.

3. A system as recited in claim 1 further comprising a guiding catheter for axial movement relative to the catheter to alternately confine the array inside the guiding catheter with the array in its first configuration, and to release the array into its second configuration.

4. A system as recited in claim 1 wherein the barrier member further comprises a base portion having a proximal end and a distal end, with the proximal end of the base portion being attached to the ring and with the plurality of deflectable extension members being integral with the distal end of the base portion.

5. A system as recited in claim 4 wherein the base portion of the barrier member is deformable when the balloon is inflated.

6. A system as recited in claim 4 wherein the barrier member in the second configuration of the array assumes a pattern selected from a group comprising a web-like pattern, a fan-like pattern, and a pattern of elongated extension arms.

7. A system as recited in claim 1 further comprising a guide wire, wherein the guide wire is pre-positioned in a vasculature of a patient with the guide wire extending into the ostium of the blood vessel, and the guide wire is engaged with the catheter for advancing the catheter through the vasculature to the ostium.

8. A system as recited in claim 1 wherein the barrier member is made of a material selected from the group comprising cobalt, chromium, platinum, nitinol and stainless steel.

9. A system as recited in claim 1 wherein the array is radiopaque.

10. A method for placing a stent at the ostium of a blood vessel comprising the steps of:
pre-positioning a guide wire in the vasculature of a patient, with the guide wire extending into the ostium of the blood vessel;
advancing a catheter over the guide wire and through the vasculature to the ostium, wherein the catheter has a shaft with a proximal end and a distal end, and defines a longitudinal axis, with an inflatable balloon mounted at the distal end of the catheter and a stent positioned on the balloon, and wherein the catheter is advanced until its distal end is near the ostium;
reconfiguring a barrier member to establish an array for use as a barrier, wherein the barrier member is attached to the catheter shaft proximal the balloon and includes a plurality of deflectable extension members, with the plurality being collectively moveable between a first configuration wherein all of the extension members are aligned substantially parallel to the axis of the catheter, and a second configuration wherein all of the extension members are biased to extend substantially perpendicular to the axis from respective locations adjacent the proximal end of the stent to establish the array;
further advancing the catheter with the array in the second configuration for use of the barrier to prevent any additional advancement of the catheter into the ostium, after the stent has been positioned in the ostium;
inflating the balloon to deploy and place the stent in the ostium;
deflating the balloon;
returning the barrier member to its first configuration; and
withdrawing the catheter from the vasculature.

11. A method as recited in claim 10 wherein the reconfiguring step comprises the steps of:
providing a guiding catheter to confine the array inside the guiding catheter with the array in its first configuration; and
moving the guiding catheter in an axial direction relative to the catheter to release the array into its second configuration.

12. A method as recited in claim 11 wherein the returning step is accomplished by moving the guiding catheter in an axial direction relative to the catheter to reconfine the array in its second configuration inside the guiding catheter.

13. A method as recited in claim 11 further comprising the step of selecting a structural pattern for the array, wherein the pattern is selected from a group comprising a web-like pattern, a fan-like pattern, and a pattern of elongated extension arms.

14. A method as recited in claim 11 further comprising the step of selecting a material for manufacture of the barrier member, wherein the material is selected from the group comprising cobalt, chromium, platinum, nitinol and stainless steel.

15. A method as recited in claim 11 further comprising the step of allowing the barrier member to deform when the balloon is inflated.

16. A system for placing a stent at the ostium of a blood vessel comprising:
a means for advancing a catheter over the guide wire and through the vasculature to the ostium, wherein the catheter has a shaft with a proximal end and a distal end, and defines a longitudinal axis, with an inflatable balloon mounted at the distal end of the catheter and a stent positioned on the balloon;
a means for reconfiguring a barrier member to establish an array for use as a barrier, wherein the barrier member is attached to the catheter shaft proximal the balloon and includes a plurality of deflectable extension members, with the plurality being collectively moveable between a first configuration wherein all of the extension members are aligned substantially parallel to the axis of the catheter, and a second configuration wherein all of the extension members are biased to extend substantially perpendicular to the axis from respective locations adjacent the proximal end of the stent to establish the array; and
a means for advancing the catheter with the array in the second configuration for use of the barrier to prevent any additional advancement of the catheter into the ostium, after the stent has been positioned in the ostium.

17. A system as recited in claim 16 wherein the advancing means is a guiding catheter.

18. A system as recited in claim 16 wherein the reconfiguring means is a guiding catheter.

19. A system as recited in claim 16 further comprising a guide wire, wherein the guide wire is pre-positioned in a vasculature of a patient with the guide wire extending into the ostium of the blood vessel, and the guide wire is engaged with the catheter for advancing the catheter through the vasculature to the ostium.

20. A system as recited in claim 16 wherein the barrier member further comprises a base portion having a proximal end and a distal end, with the proximal end of the base portion being attached to the catheter shaft and with the plurality of deflectable extension members being integral with the distal end of the base portion.
